(19) United States
(12) Patent Application Publication
(10) Pub. No.: US 2014/0294917 A1
(43) Pub. Date: Oct. 2, 2014

(54) THERMOSET AND THERMOPLASTIC FIBERS AND PREPARATION THEREOF BY UV CURING

(71) Applicants: Oleg PALCHIK, Gedera (IL); Valery Palchik, Gedera (IL)

(72) Inventors: Oleg Palchik, Gedera (IL); Valery Palchik, Gedera (IL)

(21) Appl. No.: 14/117,649

(22) PCT Filed: Nov. 26, 2012
(86) PCT No.: PCT/IB2012/052399

§ 371 Date: Nov. 14, 2013

(30) Foreign Application Priority Data
May 18, 2011 (US) 61487317

(51) Int. Cl. B29D 99/00; C08F 220/28; A61K 47/32; A61K 9/70

(52) U.S. Cl. CPC B29D 99/0078; A61K 9/70; C08F 220/28; A61K 47/32
USPC 424/443; 264/470; 425/131.1; 526/320

(57) ABSTRACT

In one embodiment, this invention is directed to a method of preparing a thermoset or thermoplastic polymer fiber comprising the following sequential steps: (i) providing a monomeric or oligomeric mixture, wherein said monomeric or oligomeric mixture comprises monomers or oligomers which polymerize by radiation; (ii) optionally heating or cooling said monomeric or oligomeric mixture for obtaining optimal viscosity; (iii) pumping said monomeric or oligomeric mixture through a spinneret, die or any other nozzle type; and (iv) radiating said monomeric or oligomeric mixture with a radiation source under room temperature, wherein said thermoset or thermoplastic polymer fibers are formed.

### FIELD OF THE INVENTION

This invention provides a process for preparing thermoset and thermoplastic polymer fibers using ultraviolet curing technology and provides encapsulated, biodegradable, renewable and functional polymer fibers prepared according to the process of this invention.

### BACKGROUND OF THE INVENTION

Modern fibers industry is a big business and fibers can be found in a wide variety of applications. Polymer fibers constitute the largest part of world fiber market and are used to prepare yarns, threads, knitted and woven fabrics, non-woven fabrics, such as wipers, diapers, industrial garments, medical and health garments or filtration garments.

There are two major groups of polymer fibers, defined based on their behavior when exposed to heat: thermoplastic and thermosetting fibers. Thermoplastic polymers are normally produced in the first step and then made into products in a subsequent process. The thermoplastic materials become soft and formable when heated. The polymer melt can be formed or shaped when in this softened (melted) state. When cooled significantly below their softening point they become rigid and usable as a formed article. This type of polymer can be readily recycled by reheating it and reshaping or forming a new article. In contrast, thermosetting polymers, upon heating, won't melt, cannot be shaped or formed to any extent and will decompose upon further heating. Thermosetting polymers are made of polymer chains that cross-link with each other irreversibly, thus forming three-dimensional (interconnected) polymer structure. The formation process of this structure is known as curing. The cure may be done through heat, or through a chemical reaction or irradiation such as ultraviolet radiation. A cured thermosetting polymer is called a thermoset. Accordingly, a thermoset material cannot be melted and re-shaped after it is cured. Examples of thermosetting polymers include Bakelite, Formica and super glues. Chemically, thermoplastic polymers could be considered as a subclass of thermosetting polymers, but with crosslinking equal to zero.

Increasing environmental concerns and ongoing legislation to cut the emissions of volatile organic compounds (VOCs) have been the major driving force behind the development of radiation curing coatings over the past 30 years. Radiation curing, including ultraviolet (UV-curing) and electron beam (EB) curing technology, is now being increasingly used in various applications due to the clean and green technology that increases productivity as compared with other traditional methods of curing. This technology is now commonly utilized to perform fast drying of protective coatings, varnishes, printing inks and adhesives, and to produce the high definition images required in the manufacture of microcircuits and printing plates. Thus, use of radiation curing can be used for polymerization and provides a fast chemical reaction, spatial resolution, ambient temperature operation, solvent-free formulations and low energy consumption.

One of the applications of UV curing technology which is related to fibers is a UV coating of optical glass fibers. Generally two-layer UV coating is applied on such fibers: inner soft coating and outside hard coating. Frequently such coatings are colored, in order to distinguish different types of glass fibers. Despite coloration, UV coating lines have enormous production, allowing two-stage coating of glass at high speeds, typically above 35 m/sec (2100 m/min).

The process of producing a fibrous form from the liquid state is called fiber spinning. There are quite a few variants of the basic fiber spinning process. The most common is called melt spinning, which as the name implies means that the fiber is produced from a polymer melt. For non meltable polymers (that degrade below the melting point) solution spinning us applied. Suffice it here to mention three important types of fiber solution spinning processes: dry spinning, wet spinning and dry jet-wet spinning. In the dry spinning process the polymer solution is extruded into an evaporating gaseous stream. In the wet spinning process the solution jets are extruded into a precipitating liquid medium. In dry jet-wet spinning the extruded solution passes through an air gap before entering a coagulation bath. A more detailed descriptions is provided below:

Melt spinning. The fiber forming material is heated above its melting point (generally 200-300° C.) and the molten material is extruded through a spinneret. The liquid jets solidify into filaments in air on emerging from the spinneret holes. Melt spinning is very commonly used to make organic fibers such as nylon, polyester and polypropylene fibers.

Dry spinning. A solution of a fiber forming polymeric material in a volatile organic solvent is extruded through a spinneret into a hot environment. A stream of hot air impinges on the jets of solution emerging from the spinneret, evaporates the solvent, and leaves the solid filaments. Fibers such as acetate, acrylic, and polyurethane elastomer are obtained by dry spinning of appropriate solutions in hot air.

Wet spinning. A polymer solution in an organic solvent is extruded through holes in a spinneret into a coagulating bath (with solvent too). The jets of liquid coalesce in the coagulating bath as result of chemical or physical changes and are drawn out as a fiber. Examples of organic fibers obtained by this process include rayon and acrylic fibers.

Dry jet-wet spinning. Aramid fibers are processed by the dry jet-wet spinning process. In this process, the anisotropic solution is extruded through the spinneret holes into an air gap (about 1 cm) and then into a coagulating bath. The coagulated fibers are washed, neutralized and dried.

Several factors are common to these methods:

Before fiber formation the polymer should be liquid. It could be achieved by melting or solubilization of polymer in solvent.

Fibers should be formed from polymer.

The process of polymerization and the process of fiber formation are separate processes.

Schematically, the process of fiber formation from preformed polymer could be described in two major steps:

Polymer formation from monomers (M): M+M→pM (relatively slow process)

Spinning process: polymerM→fiber(PolymerM) (fast process)

This invention is directed to the preparation of thermoset and thermoplastic polymer fibers and nanofibers by radiation, specifically using ultraviolet and visual radiation.

### SUMMARY OF THE INVENTION

In one embodiment, this invention is directed to a method of preparing a thermoset or thermoplastic polymer fiber comprising the following sequential steps:
(i) providing a monomeric or oligomeric mixture, wherein said monomeric or oligomeric mixture comprises monomers or oligomers which polymerize by radiation;
(ii) optionally heating or cooling said monomeric or oligomeric mixture for obtaining optimal viscosity;
(iii) pumping said monomeric or oligomeric mixture through a spinneret, die or any other nozzle type; and
(iv) radiating said monomeric or oligomeric mixture with a radiation source under room temperature, wherein said thermoset or thermoplastic polymer fibers are formed.

In one embodiment, this invention is directed to a thermoset polymer fiber prepared according to the process of this invention.

In one embodiment, this invention is directed to a thermoplastic polymer fiber prepared according to the process of this invention.

In one embodiment, this invention is directed to production of numerous fibers, fabrics (woven and nonwoven), bundles or any other multi-fiber arrangement.

In one embodiment, this invention is directed to production of nanofibers, by combining known method of nanofibers formation (e.g. electrospinning) with irradiation method (e.g. UV curing) described in this invention.

In one embodiment, this invention is directed to a polymer fiber of this invention that encapsulates an active material. In another embodiment, the polymer fiber is a thermoset polymer fiber. In another embodiment, the polymer fiber is a thermoplastic polymer fiber. In another embodiment, the polymer fiber comprises a polymer fiber and an active material, wherein said active material is encapsulated in the polymer fiber. In another embodiment, the active material comprises an agrochemical material, flavoring material, soothing material, a pharmaceutical or any combination thereof. In another embodiment, this invention is directed to a polymer fiber that encapsulates an active material, prepared according to the process of this invention.

In one embodiment, this invention is directed to a biodegradable and renewable polymer fiber comprising biodegradable monomers which polymerize by radiation. In another embodiment, this invention is directed to a biodegradable and renewable polymer fiber, prepared according to the process of this invention.

In one embodiment, this invention is directed to a functional polymer fiber. In another embodiment, the polymer fiber comprises functionalized monomers. In another embodiment, the functional groups include a fluorescent probe, a protein, DNA, a pharmaceutical or a combination thereof. In another embodiment, this invention provides a functional polymer fiber prepared according to the process of this invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a schematic process for preparing thermoset and thermoplastic polymer fibers according to the process of the present invention, using ultraviolet curing technology;

FIG. 2 depicts a schematic process of preparing nanofibers according to the process of the present invention, using a combination of electrospinning and UV curing technology;

FIG. 3 depicts an optical microscope image of the thermoset nanofibers, prepared according to Example 1; and

FIG. 4 depicts a SEM image of the thermoset nanofibers, prepared according to Example 1.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides a novel process for the formation of fibers, overcoming some of the disadvantages of existing processes. The main differences between the present process and existing processes are:

Room temperature and solvent-free fiber spinning. In the process according to the present invention the fiber precursors are liquids at room temperature, therefore no melting or solvent are required for fiber spinning.

Simultaneous formation of polymer and fiber. Unlike “classics” methods of fiber spinning, the process according to the present invention starts from monomers and not polymers. Thus the separate step of polymer formation is totally eliminated.

No need for coagulation solvent. Because the process according to the present invention is activated using UV light, there is no need for a coagulating solvent (or other solvent), or evaporation of the solvent in the polymer solution, thus VOC-free fibers are produced.

Very fast process. Due to the absences of the slow preliminary step of polymer formation, the overall fiber formation process according to the present invention is very fast.

In one embodiment, this invention is directed to thermoplastic fibers. In another embodiment, this invention is directed to a thermoplastic fiber that encapsulates an active material. In another embodiment, this invention is directed to a biodegradable and renewable thermoplastic fiber. In another embodiment, this invention is directed to a functional thermoplastic fiber.

In one embodiment, this invention is directed to thermoset fibers. In another embodiment, this invention is directed to a thermoset fiber that encapsulates an active material. In another embodiment, this invention is directed to a biodegradable and renewable thermoset fiber. In another embodiment, this invention is directed to a functional thermoset fiber.

In some embodiments, this invention is directed to a method of preparing the polymer fibers of this invention. In one embodiment, this invention is directed to a method of preparing a thermoset or thermoplastic polymer fiber comprising the following sequential steps:
(i) providing a monomeric or oligomeric mixture, wherein said monomeric or oligomeric mixture comprises monomers or oligomers which polymerize by radiation; and
(ii) simultaneously pumping said monomeric or oligomeric mixture through a spinneret or die or any nozzle arrangement and radiating said pumped mixture with a radiation source under room temperature, wherein said thermoset or thermoplastic polymer fibers are formed.

In one embodiment, the polymer fibers of this invention and/or method of preparation thereof comprise and/or make use of monomers, oligomers, monomeric mixture or oligomeric mixture, photoinitiators, diluents and other additives generally used in photopolymerization processes. In another embodiment, the monomers or oligomers of this invention polymerize by radiation. In another embodiment, the monomers, oligomers, monomeric mixture or oligomeric mixture of this invention comprise acrylates, acrylic esters, polyurethane acrylates, polyester acrylates, epoxy acrylates, acrylic acid, methyl methacrylate, methacrylic esters, acrylonitrile, plant oils, unsaturated fatty acid, epoxy monomers, vinyl-ethers, isobutyl vinyl ether, thiol-enes, styrene, propylene, ethylene, urethane, alkylene monomers, or any combination thereof. In one embodiment, the term “acrylate” as used throughout the present application covers both acrylate and methacrylate functionality. Generally, epoxy groups can react with amines, phenols, mercaptans, isocyanates or acids to form the polymer fiber of this invention. In another embodiment, the epoxy monomer reacts with amine to form a polymer fiber of this invention. In another embodiment, any material that could be polymerized by radical, cationic and anionic mechanisms using radiation and specifically ultraviolet radiation, are suitable for preparing fibers of this invention.

In one embodiment, the term “UV curing” as used throughout the present application covers both ultraviolet electromagnetic radiation and visible electromagnetic radiation.

In one embodiment, the properties of the polymer fibers of this invention are determined by the monomers, oligomers, viscosity of the composition mixture and the level of crosslinking in thermoset fibers.

In one embodiment, the polymer fiber of this invention is crosslinked between 0% (thermoplastics) to 99% (fully cross-linked). In one embodiment, the thermoset polymer fiber of this invention is crosslinked less than about 99% of its crosslinking potential. In another embodiment, the thermoset polymer fiber is crosslinked less than about 75% of its crosslinking potential. In another embodiment, the thermoset polymer fiber is crosslinked in about 50%-99% of its crosslinking potential. In another embodiment, the thermoset polymer fiber is crosslinked in about 10%-50% of its crosslinking potential.

In another embodiment, the polymer fiber is a polymer fiber that encapsulates an active material. In another embodiment, the polymer fiber is a functional polymer fiber. In another embodiment, the polymer fiber is a biodegradable and renewable polymer fiber.

In one embodiment, thermoplastic fibers offer versatility and a wide range of applications. They are commonly used in food packaging because they can be rapidly and economically formed into any shape needed to fulfill the packaging function. Non limiting examples of thermoplastic fibers are: polyethylene which is used for packaging, electrical insulation, milk and water bottles, packaging film; polypropylene which is used for carpet fibers, automotive bumpers, microwave containers and prostheses; polyvinyl chloride which is used for sheathing for electrical cables; floor and wall covering; siding or automobile instrument panels.

In one embodiment, a thermoplastic fiber of this invention and method of preparation thereof comprise and/or make use of monomers, oligomers, monomeric mixture or oligomeric mixture selected from the non limiting group of acrylates, acrylic esters, polyurethane acrylates, polyester acrylates, epoxy acrylates, acrylic acid, methyl methacrylate, methacrylic esters, acrylonitrile, plant oils, unsaturated fatty acid, epoxy monomers, vinyl-ethers, isobutyl vinyl ether, thiol-enes, styrene, propylene, ethylene, urethane, alkylene monomers, or any combination thereof. In another embodiment, the thermoplastic fiber of this invention and method of preparation thereof do not include crosslinking agents. In another embodiment, the monomers used for the preparation of thermoplastic fibers possess only one radiation-curable group, thus eliminating crosslinking possibility.

In one embodiment, a thermoset fiber of this invention and method of preparation thereof comprise and/or make use of monomers, oligomers, monomeric mixture or oligomeric mixture selected from the non limiting group of acrylates, acrylic esters, polyurethane acrylates, polyester acrylates, epoxy acrylates, acrylic acid, methyl methacrylate, methacrylic esters, acrylonitrile, plant oils, unsaturated fatty acid, epoxy monomers, vinyl-ethers, isobutyl vinyl ether, thiol-enes, styrene, propylene, ethylene, urethane, alkylene monomers, or combination thereof. In another embodiment the epoxy group reacts with alcohols, vinyl ethers, polyols acid and other monomers suitable for cationic UV curing to form the polymer fiber of this invention. In another embodiment, one or several monomers or oligomers used for the preparation of thermoset fibers possess more than one radiation-curable group.

In one embodiment, this invention provides a composition mixture and methods of preparing a polymer fiber comprising monomers and/or oligomers which polymerize and cure by radiation, specifically by ultraviolet radiation. In another embodiment the monomer or oligomer of this invention comprises an ethylenic unsaturated group which polymerize via free radical polymerization. In another embodiment, the ethylenic unsaturated group is polymerized by cationic polymerization. Non limiting examples of ethylenically unsaturated groups include (meth)acrylate, styrene, vinylether, vinyl ester, N-substituted acrylamide, N-vinyl amide, maleate ester, and fumarate ester. Other functionalities contemplated by the present invention that permit polymerization upon exposure to radiation include epoxy groups, oxetane groups, as well as thiol-ene and amine-ene systems.

In one embodiment, epoxy groups polymerize through cationic polymerization, whereas the thiol-ene and amine-ene systems polymerize through radical polymerization. In another embodiment, the epoxy groups are, for example, homopolymerized. In the thiol-ene and amine-ene systems, for example, polymerization occurs between an allylic unsaturated group and a tertiary amine group or a thiol group. In another embodiment, vinylether and (meth)acrylate groups are present in the radiation-curable components of the composition mixture of this invention. In another embodiment, (meth)acrylates are present in the radiation-curable components of the composition mixture of this invention. Mixtures of mono, di-, tri-, tetra-, and higher functionalized oligomers and/or diluents can be used to achieve the desired balance of properties, wherein the functionalization refers to the number of radiation-curable groups present in the reactive component.

In another embodiment, the monomer or oligomer of this invention comprise an epoxy group. Non limiting examples of epoxy groups include: epoxy-cyclohexane, phenylepoxyethane, 1,2-epoxy-4-vinylcyclohexane, glycidylacrylate, 1,2-epoxy-4-epoxyethyl-cyclohexane, diglycidylether of polyethylene-glycol, diglycidylether of bisphenol-A, and the like.

In another embodiment, the composition mixture could contain monomers and oligomers that polymerize using radical mechanism and another group of monomers and oligomers that polymerize using cationic mechanism. Interpenetrating Network (IPN) will be a result of the polymerization of this dual-cure system.

In one embodiment, the polymer fibers of this invention and method of preparation thereof comprise and/or make use of an oligomeric mixture wherein the oligomeric mixture comprises acrylate, methacrylate, epoxy, oxetane, vinyl-ether or thiol-enes oligomers, or any combination thereof.

In another embodiment, the oligomer of this invention included in the uncured radiation-curable compositions may vary widely, and be limited according to the performance requirements of the desired fiber, and the relatively high viscosity of the oligomer. In another embodiment, the oligomer is present in the uncured compositions in an amount ranging up to about 90 wt. %. In another embodiment, the oligomer is present in the uncured compositions in an amount from about 10 wt. % to about 80 wt. %. In another embodiment, the oligomer is present in the uncured compositions in an amount from about 30 wt. % to about 70 wt. %. In another embodiment, the oligomer is present in the uncured compositions in an amount from about 40 wt. % to about 60 wt. %, based upon the total weight of the particular composition. Illustrative oligomers useful in the inventive compositions include those containing at least one ethylenically unsaturated group, meth(acrylate) group, vinyl ether group, epoxy group, oxetane groups, or any other group suitable for UV polymerization.

In one embodiment the monomeric mixture or the oligomeric mixture is referred herein as a composition mixture.

In one embodiment, the polymer fiber and method of preparation thereof comprise and/or make use of monomers, oligomers, monomeric mixture, oligomeric mixture and optionally photoinitiators, a single additive or additives combination.

In one embodiment, a diluent is added to assist in lowering the viscosity of the uncured composition mixture. In another embodiment, a diluent is added to reduce the viscosity of the oligomer of the composition mixture. In another embodiment, monomers are added as a reactive diluent.

While any number of diluents may be introduced into the fiber formulation, the reactive diluent is advantageously a low viscosity monomer or mixture of monomers having at least one radiation-curable group. Keeping in mind the foregoing functions, reactive diluents may be present in the uncured composition mixture of this invention in an amount effective to provide the composition with a viscosity within the foregoing ranges. Typically, these diluents will be present in the compositions in amounts up to about 70 wt. %. In another embodiment, from about 5 wt. % to about 60 wt. %. In another embodiment, from about 15 wt. % to about 50 wt. %, based on the total weight of the uncured composition.

In another embodiment a diluent of this invention is a monomer or mixture of monomers having an acrylate or vinyl ether group and a C₄,-C₂₀ alkyl or a polyether moiety. Non limiting examples of diluents include: hexylacrylate, 2-ethylhexylacrylate, isobomylacrylate, decylacrylate, laurylacrylate, stearylacrylate, 2-ethoxyethoxy-ethylacrylate, laurylvinylether, 2-ethylhexylvinyl ether, N-vinyl formamide, isodecyl acrylate, isooctyl acrylate, vinyl-caprolactam, N-vinylpyrrolidone, and the like, and mixtures thereof.

Another type of reactive diluent that can be used in the uncured composition mixture is a monomer having an aromatic group. Non limiting examples of reactive diluents having an aromatic group include: ethyleneglycolphenylether acrylate, polyethyleneglycolphenylether acrylate, polypropyleneglycolphenylether acrylate, and alkyl-substituted phenyl derivatives of the above monomers, such as polyethyleneglycolnonylphenylether acrylate, and mixtures thereof.

In one embodiment, the diluent of this invention or monomers/oligomers of this invention possess an allylic unsaturated group. Non limiting examples of allylic unsaturated groups include: diallylphthalate, triallyltrimellitate, triallylcyanurate, triallylisocyanurate, diallylisophthalate, and mixtures thereof. In another embodiment, a reactive diluent or monomers/oligomers of this invention possess an amine-ene functional group. Non limiting examples include: the adduct of trimethylolpropane, isophoronediisocyanate and di(m)ethylethanolamine; the adduct of hexanediol, isophoronediisocyanate and dipropylethanolamine; and the adduct of trimethylol propane, trimethylhexamethylenediisocyanate and di(m)ethylethanolamine; and mixtures thereof.

In one embodiment, a diluent used for the preparation of thermoplastic fiber possesses only one radiation-curable group. In another embodiment, a diluent suited for the preparation of thermoset fiber possesses more than one radiation-curable group

In another embodiment, a reactive diluent comprises a monomer having two or more functional groups capable of polymerization (i.e. radiation-curable group). Non limiting examples of such suitable diluents include: Cₙ, hydrocarbondioldiacrylates wherein n is an integer from 2 to 18, Cn, hydrocarbondivinylethers wherein n is an integer from 4 to 18, Cn, hydrocarbon triacrylates wherein n is an integer from 3 to 18, and the polyether analogues thereof, and the like, such as 1,6-hexanedioldiacrylate, trimethylolpropanetriacrylate, hexanedioldivinylether, triethyleneglycoldiacrylate, pentaerythritoltriacrylate, ethoxylated bisphenol-a diacrylate, and tripropyleneglycol diacrylate, and mixtures thereof.

Examples of an epoxide monomer component or diluent that may be used in an embodiment of the present invention include but not limited to a benzyl glycidyl ether, an alpha, alpha-1,4-xylyldiglycidyl ether, a bisphenol-A diglycidyl ether, cresyl glycidyl ether, an ethyleneglycol diglycidyl ether, a diethyleneglycol diglycidyl ether, a neopentylglycol diglycidyl ether, a 1,4-butanediol diglycidyl ether, a 1,4-cyclohexanedimethanol diglycidyl ether, a trimethylopropanetriol triglycidyl ether, a glycerol triglycidyl ether, a cresyl glycidyl ether, a diglycidyl phthalate, a cresol novolac epoxide, a phenol novolac epoxide, a bisphenol-A novolac epoxide, 3,4-epoxy-cyclohexylmethyl-3′,4′-epoxycyclohexanecarboxylate, bis(3,5)4-epoxy cyclohexylmethyl) adipate, limonene dioxide, 1,2-epoxydecane, epoxydodecane, 1,2,7,8-diepoxyoctane, epoxidized soybean oil, epoxidized linseed oil, epoxidized castor oil, epoxidized natural rubber, epoxidized poly(1,2-butadiene), epoxy functional silicone resins, and the like.

As mentioned previously, reactive diluents may be incorporated into the mixture primarily to counter balance the high viscosity of the oligomers. In another embodiment, the diluents of this invention lower the viscosity of the overall composition to a level sufficient to permit the composition to be drawn into fiber using the mentioned drawing equipment. Examples of suitable viscosities for the mentioned fibers compositions range from about 300 to about 300,000 centipoise at 25° C.

In another embodiment, the composition mixture of this invention optionally further includes one or more free-radical generating photoinitiators. These components are well known to those skilled in the art, and function to hasten the cure of the radiation-curable components in the mentioned compositions. Examples of suitable free radical-type photoinitiators include, but not limited to are the following: isobutyl benzoin ether; 2,4,6-trimethylbenzoyl, diphenylphosphine-oxide; 1-hydroxycyclohexylphenyl ketone; 2-benzyl-2-dimethylamino-1-(4-morpholinovhenv1)-butan-1-one; 2,2-dimethoxy-2-phenylacetophenone; perfluorinated diphenyl titanocene; 2-methyl-1-[4-(methylthio)phenyl]-2-(4-morpholinyl)-1-propanone; 2-hydroxy-2-methyl-1-phenyl propan-1-one; 4-(2-hydroxyethoxy)phenyl-2-hydroxy-2-propyl ketone dimethoxyphenylacetophenone; 1-(4-isopropylphenyl)-2-hydroxy-2-methylpropan-1-one; 1-(4-docecyl-phenyl)-2-hydroxy-2-methylpropan-1-one; 4-(2-hydroxyethoxy)phenyl-2-(2-hydroxy-2-propyl)-ketone; diethoxyphenyl acetophenone; a mixture of (2,6-dimethoxy benzoyl)-2,4,4trimethylpentylphosphine-oxide and 2-hydroxy-2-methyl-1phenyl-propan-1-one; benzophenone; 1-propanone, 2-methyl-I-1-(4-(methylthio)phenyl)-2-(4-morpholinyl); and mixtures thereof.

In another embodiment cationic photoinitiator chosen from the group consisting of a diaryl- or triarylsulfonium salt; a diaryliodonium salt; a dialkylphenacylsulfonium salt; and the like. Examples of cationic photoinitiators may be found in U.S. Pat. Nos. 4,882,201; 4,941,941; 5,073,643; 5,274,148; 6,031,014; 6,632,960; and 6,863,701, all of which are incorporated herein by reference.

The photoinitiators, if provided, may be present at levels of from about 0.1 wt. % to 10 wt. %, and advantageously from about 0.2 wt. % to about 5 wt. %, of an uncured composition mixture, based upon the weight of the composition.

In one embodiment, additives are optionally incorporated into the fiber compositions in effective amounts. The term “additive” is used herein as material being added to the monomeric or oligomeric mixture of this invention. The additives are added to alter and improve basic mechanical, physical or chemical properties. Additives are also used to protect the polymer from the degrading effects of light, heat, or bacteria; to change such polymer processing properties such as melt flow; to provide product color; and to provide special characteristics such as improved surface appearance, reduced friction, and flame retardancy. Non limiting examples of additives include one or more plasticizers, photo-sensitizer, anti-statics, antimicrobials, flame retardants, pharmaceuticals colorants such as dyes, reactive-dyes, pigments, catalysts, lubricants, adhesion promoters, wetting agents, antioxidants, stabilizers and any combination thereof. The selection and use of such additives is within the skill of the art.

In one embodiment, the additives of this invention have migrating or non-migrating behavior. In another embodiment, the migration of such additives is controlled by altering chemical and physical parameters of the additive (e.g. dipole moment), but also by altering chemical and physical parameters of the fibers. Examples of fiber parameters that could influence migration parameters of migration additives include, but not limited to crosslinking density, polarity, hydrophilic/hydrophobic ratio, hydrogen bonds, and crystallinity. In another embodiment, the additives are present in the composition mixture in the pure form or have special encapsulation system prior to the introduction into the uncured composition mixture.

In one embodiment additives are reactive with the fiber ingredients. In another embodiment these additives are inert toward fiber ingredients.

In one embodiment, this invention is directed to a method of preparing a polymer fiber comprising a step of providing a monomeric or oligomeric mixture, wherein said monomeric or oligomeric mixture comprising monomers or oligomers which polymerize by radiation. In another embodiment, the radiation includes heat, ultrasonic sound waves, gamma radiation, infrared rays, electron beam, microwaves, ultraviolet or visible light. In another embodiment, the radiation is by ultraviolet light. In another embodiment, the radiation is by visible light.

In one embodiment, the method of preparing the polymer fiber of this invention comprise a step of optional heating or cooling the monomeric or oligomeric mixture with or without additives for obtaining optimal viscosity. In another embodiment, the composition mixture with or without additives is heated to a temperature of up to 60° C. In another embodiment, the composition mixture with or without additives is at room temperature. In another embodiment, the composition mixture with or without additives is heated to a temperature of up to 100° C. In another embodiment, the composition mixture with or without additives is heated to a temperature of between 60° C. to 100° C. In another embodiment, the composition mixture with or without additives is heated to a temperature of between 30° C. to 60° C. In another embodiment, the composition mixture with or without additives is heated to a temperature of between 30° C. to 80° C. In another embodiment, the composition mixture with or without additives is cooled to a temperature of between −20° C. to room temperature.

In one embodiment, the viscosity of the composition mixture is influenced by the temperature of the uncured composition mixture. A Temperature above room temperature tends to decrease viscosity and cooling below room temperature tends to increase viscosity of the composition mixture.

In one embodiment, the method of preparing the polymer fibers of this invention is conducted at room temperature. In another embodiment this invention is directed to a method of preparing a polymer fiber comprising a step of cooling the monomeric or oligomeric mixture with or without optional additives to temperatures above solidification point of the monomer and oligomer composition.

In one embodiment, this invention is directed to a method of preparing a polymer fiber comprising a step of pumping the composition mixture through a spinneret, die or any other nozzle type. In another embodiment, the composition mixture is extruded through the spinneret, die or any other nozzle type. In another embodiment, the composition mixture is injected or pumped through the spinneret, die or any other nozzle type. Spinnerets and dies for extruding fibers are well known to those of ordinary skill in the art. As the filaments emerge from the holes in the spinneret or die, they are radiated by a radiation source to yield the polymer fiber. In another embodiment, the radiation source causes the polymerization of the monomers or oligomers.

In one embodiment only a single hole is present in the spinneret, die or any other nozzle type, thus only monofilament fiber could be produced. In another embodiment plurality of holes are present in the spinneret, die or any other nozzle type, thus producing numerous fibers, fabrics, bundles or any other multi-fiber arrangement.

Diameter of the fibers described in this invention could be influenced by many parameters, such as spinneret/die hole size, viscosity of formulations and parameters which are known to one skilled in the art.

In one embodiment the fiber of this invention is produced under the air.

In other embodiment the fiber of this invention is produced under inert atmosphere, such as nitrogen gas, argon gas or other oxygen-free gases.

In one embodiment the invention could be used for the production of nanofibers, wherein, instead of using regular spinnerets or dies, using very small die or spinnerets holes, such as used for the preparation of meltblown fibers.

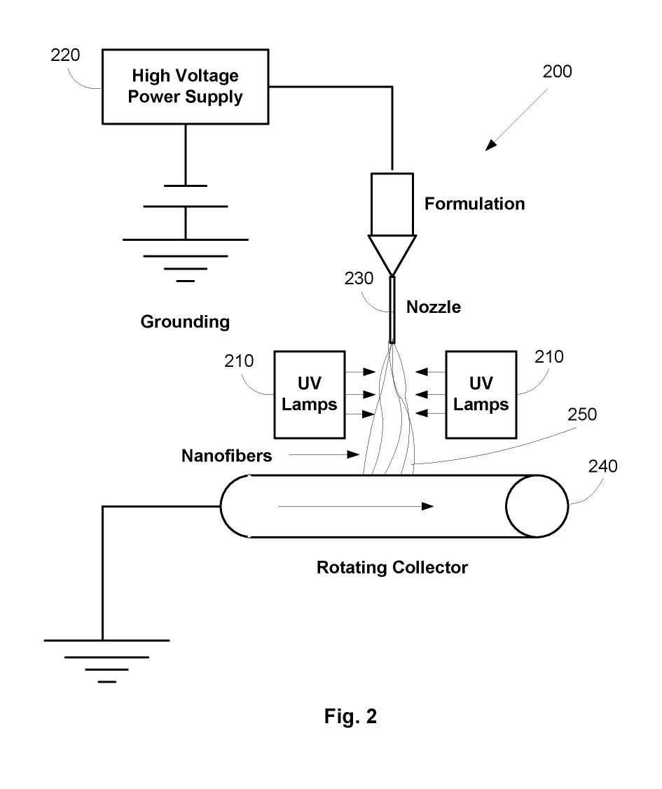

In another embodiment this invention could be combined with electrospinning method of production of nanofibers. Examples of such apparatus are shown in FIG. 2. FIG. 2 depicts a standard high-voltage nanofibers production machine 200, which is modified with UV curing units 210 (one or several). High voltage 220 is generated between the tip of the nozzle 230 (or any other known system for the formation of nanofiber structure) and a rotating collector 240, such as a conveyor for gathering nano particles or a bobbin for gathering nano filaments. Such high voltage will create continuous flow of material 250 between the nozzle tip and the collector. The presence of the UV curing units 210 in the proximity of the nozzle 230 will result in the rapid polymerization of the monomers and oligomers exiting the nozzle before they reach the collector. Such combined equipment allows for the production of nanofibers without solvents, which are extensively used in the regular electrospinning production method. Additionally, nanofibers produced using such combined apparatus can be produced at ambient temperature and do not require polymer melting, thus making possible introduction of temperature-sensitive additives into the fibers.

In one embodiment, this invention is directed to a method of preparing a polymer fiber comprising a step of radiating said monomeric or oligomeric mixture with a radiation source under room temperature, wherein polymer fibers are formed. In another embodiment, the radiation source is heat, ultra sonic sound waves, gamma radiation, infrared rays, electron beam, microwaves, ultraviolet or visible light. In another embodiment, the extruded composition is polymerized by exposure to radiation source to yield the polymer fiber of this invention. In another embodiment, the radiation source is ultraviolet light. In another embodiment, the radiation source is a visible light.

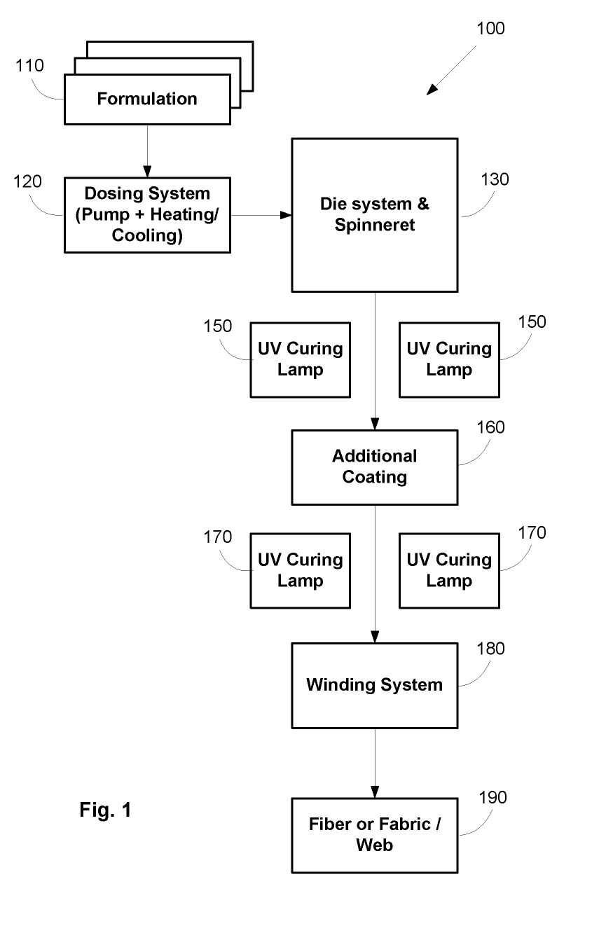

In another embodiment, the polymer fiber may be coated (see FIG. 1) by thermoplastic or thermoset polymers. Such coated fibers could be used as Polymer Optical Fibers (POFs), if the refractive index of the core and cladding are properly selected.

FIG. 1 is a block diagram describing a fiber production system 100 according to the present invention. The system comprises one or more formulation preparation tanks 110, which may optionally be heated or cooled, a dosing system 120, which may include a pump (e.g. gear pump) or piston system. Optionally the dosing system may be heated or cooled. The formulations in the dosing system may be mixed, partially mixed or remain separate, according to the type of fiber to be produced therefrom. System 100 further comprises a die system with spinnerets. The die system allows production of monolayer or multilayer fibers. The die/spinneret system 130 may be multi-hole with optional gas-blowing assistance for producing nonwoven multifilament fabrics. The multiple holes may also be used to provide encapsulating or multi-layer fibers by extruding different formulations through different holes. Also, the die/spinneret system may be used for producing short fibers by applying a chopper to the emerging fibers. UV curing units 150 are located in proximity to the spinnerets, allowing immediate polymerization of the formulation after it exits the spinneret holes. An optional additional coating system 160 allows for applying an additional UV 170 curable layer on the produced fiber. Winding system 180 consists of a capstan and a winder which allows for winding the fiber or fabric 190 onto a bobbin.

In another embodiment, the extruded composition is polymerized into different cross-sectional shapes, such as round, hollow, layers, trilobal, pentagonal or octagonal.

In one embodiment, the method of preparing polymer fibers of this invention does not include a solvent. In another embodiment the composition mixture does not include a solvent.

In one embodiment, the method of preparing polymer fibers of this invention further comprises take-up steps following the radiating step of the monomeric or oligomeric mixture with a radiation source.

Spinning take-up machines incorporate all the necessary devices to take-up, to handle and to wind the fibers emerging from the curing unit. The process involves winding filaments under varying amounts of tension over a male mould or mandrel. The mandrel rotates while a carriage moves horizontally, laying down fibers in the desired pattern. During winding the tension on the filaments can be carefully controlled. Filaments that are applied with high tension result in a final product with higher rigidity and strength; lower tension results in more flexibility. Optionally, an additional curing stage could be added after filament winding in order to preserve the obtained fiber properties by winding.

Standard take-up and winding machines could be used for fibers described in this invention.

In one embodiment, this invention is directed to a method of preparing a thermoset or thermoplastic polymer fiber which encapsulates an active material, comprising the following sequential steps:
(i) providing a monomeric or oligomeric mixture and an active material, wherein said monomeric or oligomeric mixture comprise monomers or oligomers which polymerize by radiation; and
(ii) optional heating or cooling said monomeric or oligomeric mixture for obtaining optimal viscosity;
(iii) simultaneously pumping said monomeric or oligomeric mixture through a spinneret, die or any other nozzle type and radiating said monomeric or oligomeric mixture with a radiation source under room temperature, wherein said thermoset or thermoplastic polymer fibers contain an active material inside.

In another embodiment, the active material which is encapsulated in the polymer film of this invention relates to any material that can be encapsulated and provide a unique, specific property or activity to the polymer fiber. In another embodiment, the active material includes an agrochemical material (pesticides and herbicides), flame-retardant material, flavoring/essence materials, inorganic nanoparticles, dyes, pigments, phase-change materials, odor absorbing materials, a biopolymer (enzymes), living cells, soothing materials, pharmaceuticals or any combination thereof.

The active material which is encapsulated in the polymer fiber of this invention relates to any material that can be encapsulated and provide a unique, specific property or activity to the polymer fiber.

In another embodiment, this invention is directed to a thermoset polymer fiber which encapsulates an active material and is prepared according to the process of this invention. In another embodiment, this invention is directed to a thermoplastic polymer fiber which encapsulates an active material and is prepared according to the process of this invention. In another embodiment, the optional heating step is heating to a temperature of up to 100° C.

In one embodiment, this invention is directed to a method of preparing a functional thermoset or thermoplastic polymer fiber comprising the following sequential steps:
(i) providing a monomeric or oligomeric mixture, wherein said monomeric or oligomeric mixture comprise monomers or oligomers which polymerize by radiation and said monomers or oligomers are derivatized by a functional group;
(ii) optional heating or cooling said monomeric or oligomeric mixture for obtaining optimal viscosity; and
(iii) simultaneously pumping said monomeric or oligomeric mixture through a spinneret, die or any other nozzle type and radiating said monomeric or oligomeric mixture with a radiation source under room temperature, wherein said thermoset or thermoplastic functional polymer fibers are formed.

In another embodiment, a functional group refers to any group which is covalently attached to the monomer or oligomer and provides the resulting polymer fiber a unique, specific property or activity. In another embodiment, the functional group is a fluorescent probe, an acid group, a hydroxyl group, a protein, DNA, a pharmaceutical or any combination thereof.

In another embodiment, this invention is directed to a functional thermoset polymer fiber, prepared according to the process of this invention. In another embodiment, this invention is directed to a functional thermoplastic polymer fiber, prepared according to the process of this invention. In another embodiment, the optional heating step is heating to a temperature of up to 60° C.

In one embodiment, this invention is directed to a method of preparing a biodegradable and renewable thermoset or thermoplastic polymer fiber comprising the following sequential steps:
(i) providing a monomeric or oligomeric mixture, wherein said monomeric or oligomeric mixture comprise monomers or oligomers which polymerize by radiation and said monomers or oligomers comprise an unsaturated fatty acid;
(ii) optional heating or cooling said monomeric or oligomeric mixture for obtaining optimal viscosity; and
(iii) simultaneously pumping said monomeric or oligomeric mixture through a spinneret or die or any other nozzle type and radiating said monomeric or oligomeric mixture with a radiation source under room temperature, whereby said biodegradable and renewable thermoset or thermoplastic functional polymer fibers are formed.

In one embodiment, a biodegradable and renewable polymer fiber includes monomers or oligomers which can degrade in a landfill or in a compost-like environment (i.e. biodegradable) including plant oil, or unsaturated fatty acid. In another embodiment, the monomers or oligomers are from sustainable sources such as epoxidized linseed oil, any monomer of natural origin that have ethylenical unsaturation or epoxy moiety (e.g. epoxydized fatty acids).

In another embodiment, this invention is directed to a biodegradable and renewable thermoset polymer fiber, prepared according to the process of this invention. In another embodiment, this invention is directed to a biodegradable and renewable thermoplastic polymer fiber, prepared according to the process of this invention.

The following examples are presented in order to more fully illustrate the preferred embodiments of the invention. They should in no way be construed, however, as limiting the broad scope of the invention.

### EXAMPLES

### Example 1

### Process for the Preparation of a Polymer Fiber of this Invention

For the preparation of thermoset fiber the following composition was prepared

| Material | Supplier | Type | Chemical nature | Wt % |
|---|---|---|---|---|
| CN-132 | Sartomer | aliphatic | N/A | 55.5 |
| | | diacrylate | | |
| | | oligomer | | |
| SR-9003 | Sartomer | diacrylate | Propoxylated (2) | 25.0 |
| | | monomer | neopentyl | |
| | | | glycol diacrylate | |
| SR-355 | Sartomer | tetraacrylate | Ditrimethylolpropane | 10.0 |
| | | monomer; | tetraacrylate | |
| | | crosslinker | | |
| Irgacure | Ciba | Photo- | Phosphine oxide, | 2.5 |
| 819 | | initiator | phenyl | |
| | | | bis(2,4,6-trimethyl | |
| | | | benzoyl) | |
| Darocure | Ciba | photo- | 2-Hydroxy-2-methyl-1- | 5.0 |
| 1173 | | initiator | phenyl-1-propanone | |
| Darocure | Ciba | photo- | Benzophenone | 2.0 |
| BP | | initiator | | |

All ingredients for the example 1 were mixed with gentle heating (approx 50° C.) until clear; one-phase solution was received. After the solution was cooled to room temperature, the viscosity of the solution was 620 cPs at room temperature.

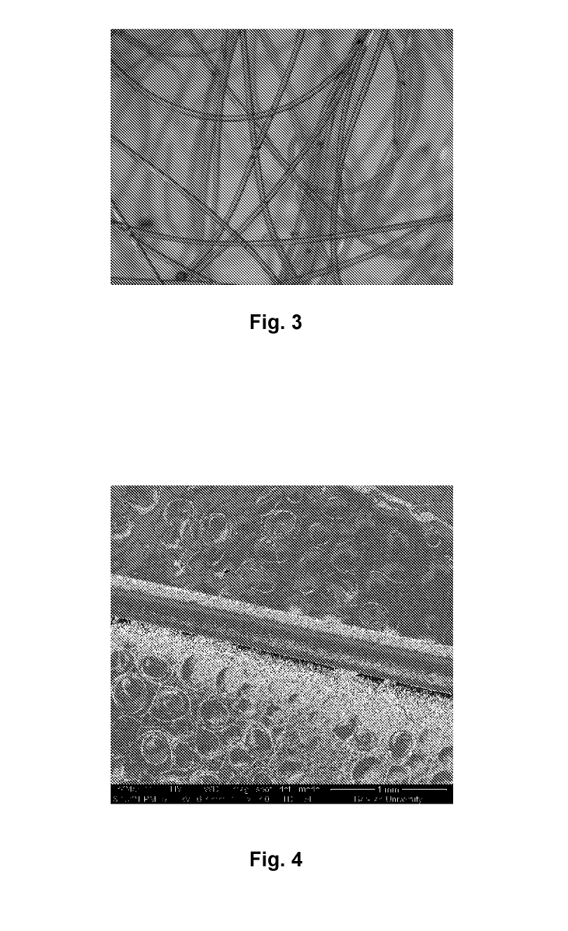

The cooled solution was poured into the flask that was connected to a die with single hole with diameter of 400 micron. Air pressure equal to 1.2 atm was applied onto the reaction mixture, which resulted in the formation of liquid jet, pouring through the hole. Three UV lamps (MHL-250, USHIO) were arranged vertically, just below the die hole. Due to the presence of UV radiation, immediate polymerization of the reaction mixture occurred, thus forming solid thermoset fiber. The fiber was collected by a two-head winder at 250 m/min speed. Optical microscope pictures and SEM pictures are presented in FIGS. 3 and 4.

### Example 2

### Process for the Preparation of a Polymer Fiber of this Invention

For the preparation of thermoset fiber the following composition was prepared

| Material | Supplier | Type | Chemical nature | Wt % |
|---|---|---|---|---|
| CN-132 | Sartomer | aliphatic | N/A | 29.4 |
| | | diacrylate | | |
| | | oligomer | | |
| SR-9020 | Sartomer | triacrylate | [001] Propoxylated | 19.6 |
| | | monomer | (3)glyceryl | |
| | | | triacrylate | |
| SR-494 | Sartomer | tetraacrylate | Ethoxylated (4) | 49.0 |
| | | monomer; | Pentaerythritol | |
| | | crosslinker | tetraacrylate | |
| Irgacure | Ciba | photo- | Phosphine oxide, | 0.5 |
| 819 | | initiator | phenyl | |
| | | | bis(2,4,6-trimethyl | |
| | | | benzoyl) | |
| Micure | Miwon | photo- | Hydroxycyclohexyl | 1.5 |
| CP4 | | initiator | phenyl ketone | |

The cooled solution was poured into the flask that was connected to a die with a single hole with diameter of 400 micron. Air pressure equal to 1.2 atm was applied onto the reaction mixture, which resulted in the formation of liquid jet, pouring through the hole. Six UV lamps (MHL-250, USHIO) were arranged vertically, just below the die hole. Due to the presence of UV radiation, immediate polymerization of the reaction mixture occurred, thus forming solid thermoset fiber. The fiber was collected by a two-head winder at 250 m/min speed.

### Example 3

### Process for the Preparation of a Polymer Fiber of this Invention

For the preparation of thermoset fiber the following composition was prepared

| Material | Supplier | Type | Chemical nature | Wt % |
|---|---|---|---|---|
| SR-415 | Sartomer | triacrylate | [002] ethoxylated | 19.6 |
| | | monomer | trimethylolpropane | |
| | | | triacrylate | |
| CN203 | Sartomer | Difunctional | N/A | 78.4 |
| | | polyester | | |
| | | acrylate | | |
| Irgacure | Ciba | photo- | Phosphine oxide, | 0.5 |
| 819 | | initiator | phenyl | |
| | | | bis(2,4,6-trimethyl | |
| | | | benzoyl) | |
| Micure | Miwon | photo- | Hydroxycyclohexyl | 1.5 |
| CP4 | | initiator | phenyl ketone | |

The cooled solution was poured into the flask that was connected to a die with a single hole with diameter of 400 micron. Air pressure equal to 1.2 atm was applied onto the reaction mixture, which resulted in the formation of liquid jet, pouring through the hole. Six UV lamps (MHL-250, USHIO) were arranged vertically, just below the die hole. Due to the presence of UV radiation, immediate polymerization of the reaction mixture occurred, thus forming solid thermoset fiber. The fiber was collected by a two-head winder at 250 m/min speed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of preparing a thermoset polymer fiber comprising the following sequential steps:
(i) providing a monomeric or oligomeric mixture, wherein said monomeric or oligomeric mixture comprise a photoinitiator and monomers or oligomers which polymerize by radiation; and
(ii) simultaneously pumping said monomeric or oligomeric mixture through a spinneret die or any other nozzle type and radiating said pumped mixture with a radiation source under room temperature, whereby said thermoset polymer fibers are formed.

2. The method of claim 1, further comprising heating or cooling said monomeric or oligomeric mixture for obtaining optimal viscosity, before said step of pumping.

3. The method of claim 1, wherein said monomeric or oligomeric mixture comprises acrylate, methacrylate, epoxy, vinyl-ether, thiol containing compound, allyl containing compound, any other unsaturated compound, or any combination thereof.

4. (canceled)

5. The method of claim 1, wherein said radiation source is ultraviolet radiation, visible radiation or combination thereof, said nozzle has a plurality of holes, or combination thereof.

6. The method of claim 1, wherein said method does not include any solvent.

7. The method of claim 1, wherein said method further comprises a winding step following radiating said monomeric or oligomeric mixture with a radiation source.

8. The method of claim 1, wherein said monomeric or oligomeric mixture comprises an active material, yielding a polymer fiber which encapsulates an active material.

9. The method of claim 8, wherein said active material is an agrochemical material, fragrance material, a flavoring material, a biopolymer (enzymes), living cells, a soothing material, a pharmaceutical or any combination thereof.

10. The method of claim 1, wherein said monomers or oligomers are derivatized to include different chemical functional groups and form a polymer fiber with chemically functionalized surface.

11. The method of claim 10, wherein said functional groups are capable of attaching to a fluorescent probe, a protein, DNA, a pharmaceutical or a combination thereof.

12. A polymer fiber which encapsulates an active material, prepared according to the method of claim 8.

13. (canceled)

14. (canceled)

15. A biodegradable and renewable thermoset polymer fiber, prepared according to the process of claim 1.

16. (canceled)

17. The polymer fiber of claim 15, wherein said monomers comprises plant oil, unsaturated fatty acid or derivatives thereof.

18. An apparatus for preparing a thermoset polymer fiber comprising:
a. one or more formulation tanks comprising one or more mixtures of monomers or oligomers which polymerize by radiation;
b. a dosing system configured to receive said one or more formulations;
c. a die and spinneret system comprising one or more holes and configured to receive said one or more formulations from said dosing system and pump them through said one or more holes; and
d. one or more UV curing lamps configured to cure said injected material, thereby creating a thermoset polymer fiber.

19. (canceled)

20. The apparatus of claim 18, wherein said one or more formulations comprise at least two formulations and wherein said one or more holes comprise a plurality of holes, each one of said plurality of holes configured to receive one of said at least two formulations.

21.-26. (canceled)

27. The method of claim 1, wherein at least one of said monomers or oligomers possess more than one radiation-curable group.

28. A thermoset polymer fiber which encapsulates an active material comprising a thermoset polymer and an active material, wherein said active material comprises an agrochemical material, flavoring material, soothing material, a pharmaceutical or any combination thereof.

29. The apparatus of claim 18, further comprising a winding system configured to wind said polymer fibers, a high-voltage power supply connected to said apparatus, a coating system configured to apply an additional UV curable layer on the produced fiber, or any combination thereof.
